(19) United States
(12) Patent Application Publication
(10) Pub. No.: US 2024/0342361 A1
(43) Pub. Date: Oct. 17, 2024

(54) DUAL-BARREL INJECTOR AND ASSOCIATED SYSTEMS AND METHODS

(71) Applicant: Massachusetts Institute of Technology, Cambridge, MA (US)

(72) Inventors: Kripa K. Varanasi, Lexington, MA (US); Vishnu Jayaprakash, Cambridge, MA (US)

(73) Assignee: Massachusetts Institute of Technology, Cambridge, MA (US)

(21) Appl. No.: 18/294,658

(22) PCT Filed: Apr. 11, 2022
(86) PCT No.: PCT/US2022/024234

§ 371 Date: Feb. 2, 2024

Related U.S. Application Data
(60) Provisional application No. 63/229,113, filed on Aug. 4, 2021.

(51) Int. Cl. A61M 5/14; A61M 5/19; A61M 5/20; A61M 5/315

(52) U.S. Cl. CPC A61M 5/1408; A61M 5/19; A61M 5/2066; A61M 2005/31598

(57) ABSTRACT

Articles such as dual-barrel injectors are generally described. Associated systems and methods are also described. In some embodiments, the articles are useful for injecting viscous fluids, such as concentrated drug formulations. In certain embodiments, the article comprises a first conduit (e.g., for transporting a viscous fluid) and a second conduit (e.g., for transporting a lubricating fluid). In some embodiments, the fluid from the second conduit (e.g., a lubricating fluid) lubricates the flow of the fluid from the first conduit (e.g., a viscous drug) by surrounding the fluid from the first conduit, and the lower viscosity of the fluid from the second conduit allows the fluid from the first conduit to flow more easily through the system.

### RELATED APPLICATIONS

This application claims priority under 35 U.S.C. § 119(e) to U.S. Provisional Patent Application No. 63/229,113, filed Aug. 4, 2021, which is hereby incorporated by reference in its entirety.

### TECHNICAL FIELD

Articles such as dual-barrel injectors are generally described. Associated systems and methods are also described.

### SUMMARY

Articles such as dual-barrel injectors are generally described. Associated systems and methods are also described. In some embodiments, the articles are useful for injecting viscous fluids, such as concentrated drug formulations. In certain embodiments, the article comprises a first conduit (e.g., for transporting a viscous fluid) and a second conduit (e.g., for transporting a lubricating fluid). In some cases, the first conduit is fluidically connected to a first internal volume of a chamber and the second conduit is fluidically connected to a second internal volume of the chamber, wherein the second internal volume surrounds at least a portion of the first internal volume. In certain instances, the chamber (e.g., the first internal volume and/or the second internal volume of the chamber) is in fluidic communication with a needle. According to some embodiments, the article comprises a first plunger at least partially disposed in the first conduit and a second plunger at least partially disposed in the second conduit. In accordance with certain embodiments, the article is configured such that when the first plunger and the second plunger are compressed, fluid from the first conduit is transported to the chamber and/or needle and fluid from the second conduit is transported to the chamber and/or needle such that the fluid from the second conduit at least partially (e.g., partially or completely) axially surrounds fluid from the first conduit (e.g., in the chamber, at an outlet(s) of the chamber, and/or in a needle). In some embodiments, the fluid from the second conduit (e.g., a lubricating fluid) lubricates the flow of the fluid from the first conduit (e.g., a viscous drug) by surrounding the fluid from the first conduit, and the lower viscosity of the fluid from the second conduit allows the fluid from the first conduit to flow more easily through the system. In some embodiments, the fluid from the second conduit preferentially wets, relative to the fluid from the first conduit, the interior surface of the needle and/or chamber through which the fluids are transported. In some cases, the fluid from the first conduit does not contact and/or does not substantially contact the interior surface of the needle and/or chamber through which the fluid from the first conduit is transported. The subject matter of the present disclosure involves, in some cases, interrelated products, alternative solutions to a particular problem, and/or a plurality of different uses of one or more systems and/or articles.

Certain embodiments relate to articles (e.g., articles for delivery of a fluid). In some embodiments, the article for delivery of a fluid comprises a first conduit comprising an inlet and an outlet; a second conduit comprising an inlet and an outlet, the first conduit and the second conduit arranged in a side-by-side configuration; and a chamber comprising: a first internal volume; a first inlet fluidically connected to the outlet of the first conduit and the first internal volume of the chamber; a second internal volume that surrounds at least a portion of the first internal volume; and a second inlet fluidically connected to the outlet of the second conduit and the second internal volume of the chamber.

In certain embodiments, the article for delivery of a fluid comprises a first conduit; a first plunger associated with the first conduit; a second conduit in a side-by-side configuration with the first conduit; a second plunger associated with the second conduit; a solid body connecting the first plunger and the second plunger; and a chamber in fluidic communication with the first conduit and the second conduit; wherein the article is configured such that when the first plunger and the second plunger are compressed, fluid within the first conduit is transported to the chamber and fluid within the second conduit is transported to the chamber such that the fluid from the second conduit at least partially (e.g., partially or completely) axially surrounds fluid from the first conduit.

Certain embodiments relate to fluidic elements. In some embodiments, the fluidic element comprises a first internal volume; a second internal volume, wherein the second internal volume surrounds at least a portion of the first internal volume; a first inlet within a surface of the fluidic element, the first inlet in fluidic communication with the first internal volume; a second inlet within the surface of the fluidic element, the second inlet in fluidic communication with the second internal volume; and an outlet in fluidic communication with the first internal volume and the second internal volume.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is, in accordance with some embodiments, a cross-sectional schematic illustration of an article comprising a first conduit, a second conduit, and a chamber comprising a first internal volume and a second internal volume.

FIG. 1B is, in accordance with some embodiments, a cross-sectional schematic illustration of a chamber comprising a second internal volume axially surrounding a first internal volume.

FIG. 1C is, in accordance with some embodiments, a cross-sectional schematic illustration of an article comprising a first conduit and a second conduit at least partially contained within the same housing and a chamber comprising a first internal volume and a second internal volume.

FIG. 1D is, in accordance with some embodiments, a cross-sectional schematic illustration of an article comprising a first plunger, a second plunger, a first conduit, a second conduit, a chamber comprising a first internal volume and a second internal volume, and a needle.

FIG. 1E is, in accordance with some embodiments, a cross-sectional schematic illustration of an article comprising a first plunger, a second plunger, a solid body connecting the first plunger and the second plunger, a first conduit, a second conduit, and a chamber comprising a first internal volume and a second internal volume.

FIG. 1F is, in accordance with some embodiments, a cross-sectional schematic illustration of a fluidic element comprising a first internal volume and a second internal volume.

FIG. 2A is, in accordance with some embodiments, a perspective view schematic illustration of an article, wherein the top dotted line rectangle shows a top portion of the article, which may be provided fully assembled (e.g., as shown in FIG. 2A) or may be attached to conventional syringes, in some embodiments.

FIG. 2B is, in accordance with some embodiments, a schematic illustration of the portion of the article of FIG. 2A shown in the top dotted line rectangle of FIG. 2A.

FIG. 2C is, in accordance with some embodiments, a schematic illustration of the portion of the article of FIG. 2B shown in the dotted line rectangle of FIG. 2B. FIG. 2C shows, in accordance with certain embodiments, some dimensions that can be controlled during manufacturing.

FIG. 3 is, in accordance with certain embodiments, a schematic illustration of a droplet on a surface within a medium, which can be used to illustrate how the spreading coefficient is determined.

### DETAILED DESCRIPTION

Articles such as dual-barrel injectors are generally described. Associated systems and methods are also described. In some embodiments, injectability of a viscous fluid, such as a concentrated drug formulation, is desired. However, the non-linear relationship between formulation concentration and viscosity can greatly limit the ability to inject high concentration drug formulations, which are frequently needed for biologics and/or subcutaneous administration. As drug concentrations increase over 50 mg/mL, the corresponding viscosities frequently range from 20 cP to 1000 cP, making injection through conventional delivery methods (e.g., syringes) extremely challenging. For example, high hydraulic resistance presented by flow through needles at such high concentrations frequently induces large back pressures. In some embodiments, the articles, systems, and/or methods described herein reduce these resistances and enhance the injectability of such high concentration drug formulations, and other high viscosity fluids, by achieving axially lubricated flow with the fluid of interest (e.g., a fluid from the first conduit) and a lubricating fluid (e.g., a fluid from the second conduit).

However, axially lubricated flow can be very difficult to achieve in practical systems. For example, eccentricity frequently arises if the densities of the fluids are not substantially the same, such that the fluid of interest (e.g., a fluid from the first conduit) contacts the interior surface of the needle and/or chamber, reducing the lubrication effect from the lubricating fluid (e.g., a fluid from the second conduit). However, trying to match the densities of the fluids can be extremely impractical in many cases. Avoiding eccentricity can be especially difficult in cases where the fluids are miscible. While vertical operation could be used to avoid eccentricity in certain cases, this is also typically impractical, as most subcutaneous injections are not administered vertically. Moreover, vertical operation would only facilitate injection of miscible fluids, in certain cases, and would typically not work with immiscible fluids. Certain of the articles, systems, and methods disclosed herein are capable of achieving axially lubricated flow in practical systems, despite these challenges. In some embodiments, the articles, systems, and methods disclosed herein include concepts disclosed in International Patent Application Number PCT/US2021/015397, filed Jan. 28, 2021, and published as International Patent Application Publication No. WO 2021/154927 on Aug. 5, 2021, which is hereby incorporated by reference in its entirety.

In certain embodiments, the article comprises a first conduit (e.g., for transporting a viscous fluid, such as a concentrated drug) and a second conduit (e.g., for transporting a lubricating fluid). In some cases, the first conduit is fluidically connected to a first internal volume of a chamber and the second conduit is fluidically connected to a second internal volume of the chamber, wherein the second internal volume surrounds at least a portion of the first internal volume. The chamber can be located downstream of the first conduit and the second conduit. In certain instances, the chamber (e.g., the first internal volume and/or the second internal volume) is in fluidic communication with a needle. The needle can be located downstream of the chamber and downstream of the first conduit and the second conduit.

According to some embodiments, the article comprises a first plunger at least partially disposed in the first conduit and a second plunger at least partially disposed in the second conduit. In accordance with certain embodiments, the article is configured such that when the first plunger and the second plunger are compressed, fluid from the first conduit is transported to the chamber and/or needle and fluid from the second conduit is transported to the chamber and/or needle such that the fluid from the second conduit at least partially axially surrounds fluid from the first conduit (e.g., in the chamber, at an outlet(s) of the chamber, and/or in a needle). According to certain embodiments, fluid from the second conduit at least partially (e.g., at least 10%, at least 25%, at least 50%, at least 75%, at least 90%, or fully) axially surrounds fluid from the first conduit throughout at least a portion (e.g., at least 10%, at least 25%, at least 50%, at least 75%, at least 90%, or fully) of the length of the needle. For example, in some cases, fluid from the second conduit fully surrounds fluid from the first conduit throughout the entire length of the needle. In some embodiments, the fluid from the second conduit (e.g., a lubricating fluid) lubricates the flow of the fluid from the first conduit (e.g., a viscous drug) by surrounding the fluid from the first conduit, and the lower viscosity of the fluid from the second conduit allows the fluid from the first conduit to flow more easily through the system. In some embodiments, the fluid from the second conduit (e.g., a lubricating fluid) lubricates the flow of the fluid from the first conduit (e.g., a viscous drug) by preferentially wetting, relative to the fluid from the first conduit, the interior surface of the needle and/or chamber through which the fluids are transported. In some cases, the fluid from the first conduit does not contact and/or does not substantially contact the interior surface of the needle and/or chamber through which the fluid from the first conduit is transported.

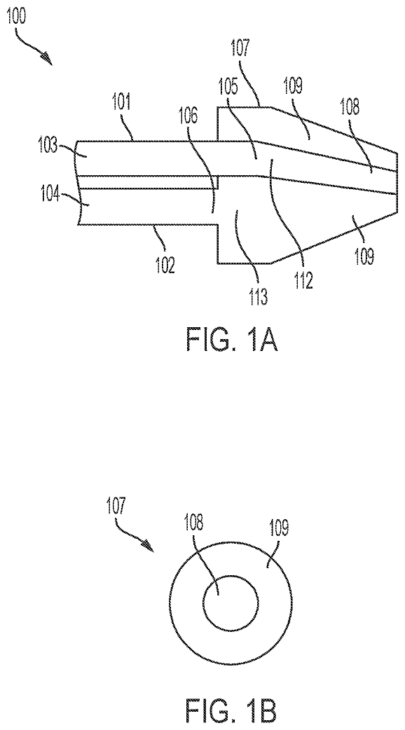

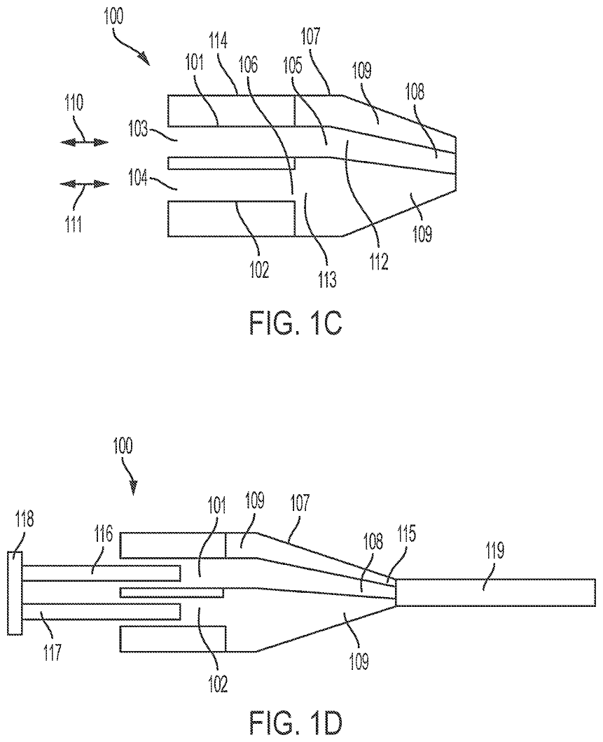

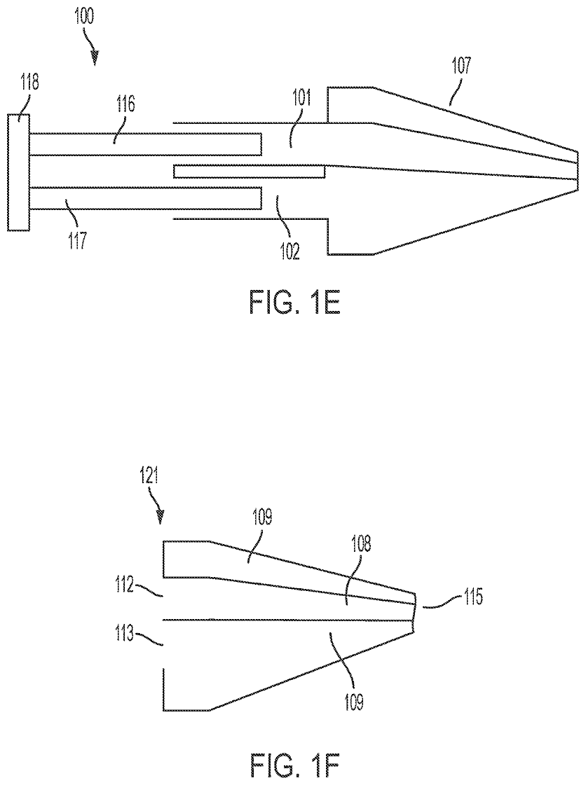

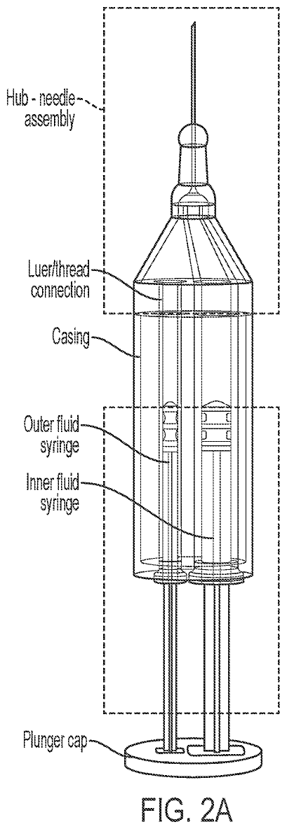

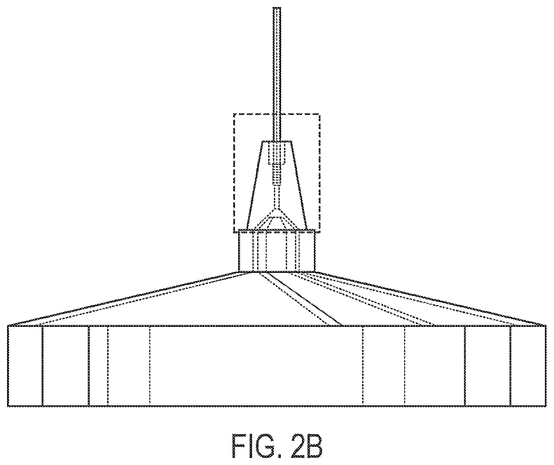

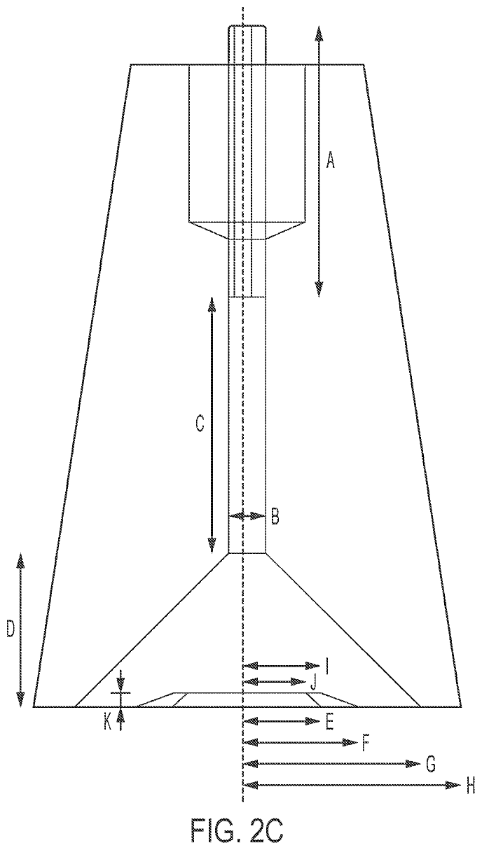

Certain embodiments are related to articles. Some such articles are illustrated schematically in FIGS. 1A-1F. In some embodiments, the article is an article for delivery of a fluid (e.g., a pharmaceutical, such as a biologic).

In certain embodiments, the article comprises a first conduit. For example, in FIG. 1A, in some embodiments, article 100 comprises first conduit 101. In some instances, the first conduit comprises an inlet and/or an outlet. For example, in FIG. 1A, in certain embodiments, first conduit 101 comprises inlet 103 and outlet 105. In some embodiments, the first conduit is configured to contain and/or transport a first fluid (e.g., the desired fluid to be delivered, such as a viscous fluid). In some cases, the fluid from the first conduit comprises a drug, a monoclonal antibody, an enzyme, a peptide, a recombinant therapeutic protein, a biologic, a bone putty, a hydrogel, cells, a gel, a bio-ink, a suspension-based formulation, and/or a biopharmaceutical. For example, in certain embodiments, the fluid comprises a concentrated drug formulation (e.g., biologic).

In some embodiments, the article comprises a second conduit. For example, in FIG. 1A, in some embodiments, article 100 comprises second conduit 102. In certain cases, the second conduit comprises an inlet and/or an outlet. For example, in FIG. 1A, in certain embodiments, second conduit 102 comprises inlet 104 and outlet 106. In some embodiments, the second conduit is configured to contain and/or transport a second fluid (e.g., a lubricating fluid). According to certain embodiments, the second fluid has a lower viscosity than the first fluid. In some cases, the second fluid (e.g., the lubricating fluid) comprises water, a buffer (e.g., a pharmaceutically acceptable buffer, such as a buffer used in a pharmaceutical product, such as a biologic), a formulation (e.g., a pharmaceutical formulation, such as a biologic formulation), a water-based solution, saline, a biocompatible oil (e.g., squalene, a fluorinated oil (e.g., HFE-7500), mineral oil, and/or triglyceride oil), benzyl benzoate, a metabolizable oil, an immunologic adjuvant (e.g., MF59, AS02, AS03 and/or AS04), and/or safflower oil.

In some embodiments, the first fluid and the second fluid comprise completely different components. For example, in some embodiments, the first fluid and the second fluid do not have any components in common. One such example would be if the first fluid comprises a drug and water, while the second fluid comprises an organic solvent.

In some embodiments, the first fluid and the second fluid comprise one or more components (e.g., a solvent and/or a buffer) that are the same. For example, in certain embodiments, the first fluid and the second fluid both comprise water.

In certain embodiments, the first fluid and/or the second fluid comprises one or more components that are different. For example, in some embodiments, the first fluid comprises water and the second fluid does not.

In certain embodiments, the first fluid and the second fluid comprise one or more components that are different and one or more components that are the same. For example, in some embodiments, the first fluid and the second fluid comprise the same components except that the first fluid also has a drug (e.g., a biologic). For example, in certain embodiments, the first fluid and the second fluid both comprise water, but the first fluid has a drug (e.g., a biologic) and the second fluid does not. In some embodiments, the first fluid and the second fluid comprise exactly the same components (e.g., a buffer) except that one of the fluids (e.g., the first fluid) has an additional component (e.g., a drug).

In some embodiments, the first fluid and the second fluid comprise exactly the same components (e.g., a buffer and a drug), but the concentrations of one or more of the components are different (e.g., a drug). For example, in some embodiments, the first fluid and the second fluid comprise exactly the same components (e.g., a buffer and a drug), but the concentration of one or more of the components (e.g., a drug) is higher in the first fluid. As a skilled person would understand, in some embodiments, the different concentration of one or more of the components could result in different physical and/or chemical properties. For example, in an embodiment where the first fluid has a high concentration of a biologic drug and the second fluid has a low concentration of the biologic drug, but the first and second fluids are otherwise identical, the viscosity and/or density of the first fluid may be much higher than that of the second fluid.

In some embodiments, the molar concentration of one component (e.g., a drug) in the second fluid is greater than or equal to 5%, greater than or equal to 10%, greater than or equal to 20%, greater than or equal to 30%, greater than or equal to 40%, greater than or equal to 50%, greater than or equal to 60%, greater than or equal to 75%, greater than or equal to 90%, or greater than or equal to 95% less than the molar concentration of that component in the first fluid. In some embodiments, the molar concentration of one component (e.g., a drug) in the second fluid is less than or equal to 100%, less than or equal to 99%, less than or equal to 95%, less than or equal to 90%, less than or equal to 80%, less than or equal to 70%, less than or equal to 60%, or less than or equal to 50% less than the molar concentration of that component in the first fluid. Combinations of these ranges are also possible (e.g., greater than or equal to 5% and less than or equal to 100%, or greater than or equal to 10% and less than or equal to 50%). For example, if the molar concentration of the component was 1M in the first fluid and 0.1M in the second fluid, the molar concentration of the component in the second fluid would be 90% less than that in the first fluid.

According to certain embodiments, the first conduit and the second conduit are arranged in a side-by-side configuration. For example, in FIG. 1C, in some embodiments, first conduit 101 and second conduit 102 are arranged in a side-by-side configuration. In some cases, the first conduit comprises a longitudinal axis, the second conduit comprises a longitudinal axis, and at least a portion (e.g., at least 10%, at least 25%, at least 50%, at least 75%, at least 90%, or all) of the longitudinal axis of the first conduit is within 10 degrees (e.g., within 5 degrees, within 2 degrees) of parallel (or is parallel) to at least a portion (e.g., at least 10%, at least 25%, at least 50%, at least 75%, at least 90%, or all) of the longitudinal axis of the second conduit. For example, in FIG. 1C, in some embodiments, first conduit 101 comprises longitudinal axis 110 and second conduit 102 comprises longitudinal axis 111, and longitudinal axis 110 is within 10 degrees (0 degrees, in this case) of parallel to longitudinal axis 111.

According to some embodiments, the first conduit and the second conduit are each at least partially (e.g., at least 10%, at least 25%, at least 50%, at least 75%, at least 90%, or fully) contained within the same housing. For example, in FIG. 1C, in some embodiments, first conduit 101 and second conduit 102 are at least partially (in this case, fully) contained within housing 114.

In accordance with some embodiments, the article comprises a chamber. For example, in FIG. 1A, in certain embodiments, article 100 comprises chamber 107. In certain instances, the chamber comprises a first internal volume. For example, in FIG. 1A, in certain instances, chamber 107 comprises first internal volume 108. In some cases, the chamber comprises a first inlet. For example, in FIG. 1A, in some cases, chamber 107 comprises first inlet 112. According to certain embodiments, the first inlet is fluidically connected to the outlet of the first conduit and/or the first internal volume of the chamber. For example, in FIG. 1A, in accordance with some embodiments, first inlet 112 is fluidically connected to outlet 105 of first conduit and first internal volume 108 of chamber 107.

In some embodiments, the chamber comprises a second internal volume. For example, in FIG. 1A, according to certain embodiments, chamber 107 comprises second internal volume 109 (including both the portion above first internal volume 108 and the portion below first internal volume 108). In accordance with some embodiments, the portion of second internal volume 109 above first internal volume 108 and the portion of second internal volume 109 below first internal volume 108 are fluidically connected, such that fluid within second internal volume 109 surrounds the fluid within first internal volume 108. In certain cases, the second internal volume surrounds at least a portion of (e.g., at least 10%, at least 25%, at least 50%, at least 75%, at least 90%, or all) of the first internal volume. For example, in FIG. 1A, in some instances, second internal volume 109 surrounds at least a portion of first internal volume 108. As another example, in FIG. 1B (a cross-sectional view of chamber 107), second internal volume 109 surrounds all of first internal volume 108. In some instances, the chamber comprises a second inlet. For example, in certain cases, chamber 107 comprises second inlet 113. In certain embodiments, the second inlet is fluidically connected to the outlet of the second conduit and/or the second internal volume of the chamber. For example, in some embodiments, second inlet 113 is fluidically connected to outlet 106 of second conduit 102 and/or to second internal volume 109 of chamber 107.

In certain embodiments, the chamber comprises one or more outlets. For example, in FIG. 1D, in accordance with some embodiments, chamber 107 comprises outlet 115. In some cases, the outlet(s) is fluidically connected to the first internal volume and/or the second internal volume. For example, in FIG. 1D, in some embodiments, outlet 115 of chamber 107 is fluidically connected to first internal volume 108 and/or second internal volume 109 of chamber 107. In certain instances, outlet 115 comprises an outlet for first internal volume 108 and an outlet for second internal volume 109. In some such cases, the outlet of the second internal volume surrounds the outlet of the first internal volume.

According to certain embodiments, the chamber is configured to convert the flow of fluid from side-by-side (e.g., within 10 degrees (e.g., within 5 degrees, within 2 degrees) of parallel (or parallel)) flow to concentric flow.

In accordance with some embodiments, the chamber comprises a connector that connects two or more components of the article (e.g., the first conduit, the second conduit, and/or the needle). For example, in some cases, the chamber connects both the first conduit and the second conduit to a needle.

As another example, in FIG. 1E, in some instances, article 100 comprises chamber 107 (e.g., a connector). In certain cases, the chamber is in fluidic communication with the first conduit and/or the second conduit. For example, in FIG. 1E, in certain embodiments, chamber 107 is in fluidic communication with first conduit 101 and/or second conduit 102. In some instances, the article is configured such that when the first plunger and the second plunger are compressed, fluid within the first conduit is transported to the chamber (and/or needle) and fluid within the second conduit is transported to the chamber (and/or needle) such that the fluid from the second conduit at least partially (e.g., at least 10%, at least 25%, at least 50%, at least 75%, at least 90%, or fully) axially surrounds fluid from the first conduit (e.g., in the chamber, at an outlet(s) of the chamber, and/or in a needle). For example, in FIG. 1E, article 100 is configured such that when first plunger 116 and second plunger 117 are compressed, fluid within first conduit 101 is transported to chamber 107 and fluid within second conduit 102 is transported to chamber 107, such that the fluid from second conduit 102 at least partially (e.g., partially or completely) axially surrounds fluid from first conduit 101. As another example, in FIG. 1D, article 100 is configured such that when first plunger 116 and second plunger 117 are compressed, fluid within first conduit 101 is transported to needle 119 and fluid within second conduit 102 is transported to needle 119, such that the fluid from second conduit 102 at least partially (e.g., partially or completely) axially surrounds fluid from first conduit 101. A second fluid is said to “axially surround” a first fluid when a continuous pathway can be traced, within the second fluid, that surrounds the longitudinal axis of the first fluid. When a second fluid is said to partially (e.g., partially or completely) axially surround (or axially surround a portion of) the first fluid, the second fluid axially surrounds the first fluid over at least a portion of the longitudinal axis of the first fluid.

According to some embodiments, the article comprises a first plunger. For example, in FIG. 1D, in certain instances, article 100 comprises first plunger 116. In some cases, the first plunger is associated with the first conduit. For example, in certain cases, the first plunger is at least partially (e.g., at least 10%, at least 25%, at least 50%, at least 75%, at least 90%, or fully) disposed in the first conduit. For example, in FIG. 1D, in some embodiments, first plunger 116 is at least partially disposed in first conduit 101.

According to certain embodiments, the article comprises a second plunger. For example, in FIG. 1D, in some embodiments, article 100 comprises second plunger 117. In certain cases, the second plunger is associated with the second conduit. For example, in some instances, the second plunger is at least partially (e.g., at least 10%, at least 25%, at least 50%, at least 75%, at least 90%, or fully) disposed in the second conduit. For example, in FIG. 1D, in some embodiments, second plunger 117 is at least partially disposed in second conduit 102.

In accordance with some embodiments, the first plunger and the second plunger are connected by a solid body. For example, in FIG. 1D, in certain embodiments, first plunger 116 and second plunger 117 are connected by solid body 118. In certain instances, the solid body comprises a cap connected to an end of the first plunger and an end of the second plunger. For example, in FIG. 1D, in some cases, solid body 118 comprises a cap connected to an end of first plunger 116 and an end of second plunger 117. As would be understood by those of ordinary skill in the art, a solid body is a body that includes a solid component. Solid bodies may, in certain cases, include cavities and/or be hollow, as long as other portions of the solid body are made of solid material. In some embodiments, the first plunger, the second plunger, and the solid body are all part of a single component made of the same material. In other embodiments, the first plunger, the second plunger, and/or the solid body can be made of different materials and assembled together. Other configurations are also possible.

In accordance with certain embodiments, the article comprises a needle. For example, in FIG. 1D, in some embodiments, article 100 comprises needle 119. In some cases, the needle is in fluidic communication with the first internal volume and/or the second internal volume. For example, in FIG. 1D, in certain embodiments, needle 119 is in fluidic communication with first internal volume 108 and second internal volume 109. The needle may be directly connected to the chamber, first internal volume, and/or second internal volume (e.g., with nothing in between) or it may be indirectly connected to the chamber, first internal volume, and/or second internal volume (e.g., with an additional chamber in between).

In certain cases, the article comprises a fluidic element (e.g., a connector and/or a chamber disclosed herein).

In some embodiments, the fluidic element comprises a first internal volume (e.g., any first internal volume disclosed herein). For example, in FIG. 1F, in some instances, fluidic element 121 comprises first internal volume 108. In certain embodiments, the fluidic element comprises a second internal volume (e.g., any second internal volume disclosed herein). For example, in FIG. 1F, in some instances, fluidic element 121 comprises second internal volume 109. In some instances, the second internal volume surrounds at least a portion (e.g., at least 10%, at least 25%, at least 50%, at least 75%, at least 90%, or all) of the first internal volume. For example, in FIG. 1F, in certain embodiments, second internal volume 109 surrounds at least a portion of first internal volume 108.

In certain embodiments, the fluidic element comprises a first inlet (e.g., any first inlet disclosed herein). For example, in FIG. 1F, in some embodiments, fluidic element 121 comprises first inlet 112. In some cases, the first inlet is within a surface of the fluidic element. In certain instances, the first inlet is in fluidic communication with the first internal volume. For example, in FIG. 1F, in certain embodiments, first inlet 112 is in fluidic communication with first internal volume 108.

In some embodiments, the fluidic element comprises a second inlet (e.g., any second inlet disclosed herein). For example, in FIG. 1F, in some embodiments, fluidic element 121 comprises second inlet 113. In some cases, the second inlet is within a surface of the fluidic element. In certain instances, the second inlet is in fluidic communication with the second internal volume. For example, in FIG. 1F, in certain embodiments, second inlet 113 is in fluidic communication with second internal volume 109.

According to certain embodiments, the fluidic element comprises an outlet. For example, in FIG. 1F, in some cases, fluidic element 121 comprises outlet 115. In some instances, the outlet is in fluidic communication with the first internal volume and/or the second internal volume. For example, in FIG. 1F, in certain instances, outlet 115 is in fluidic communication with first internal volume 108 and/or second internal volume 109.

In certain embodiments, the article comprises luer and/or threaded connectors (e.g., between components, such as between the chamber and the first conduit and/or second conduit, between the chamber and a needle, or between the fluidic element and a commercially available syringe and/or needle).

Certain of the embodiments disclosed herein can provide one or more of several benefits, including reduced contamination, reduced needle clogging, reduced protein inactivation (e.g., when the first fluid comprises a protein), increased concentrations of formulations (e.g., the first fluid may be a high concentration drug formulation), increased viscosity of fluids, increased feasibility of subcutaneous administration (rather than intravenous administration), smaller needles, shorter injection times, reduced pain, fewer doses, reduced hydrodynamic resistance in the needle, reduced shear forces on the first fluid (e.g., viscous fluid), reduced pressures, ease of manufacture (e.g., as the chamber, connector, and/or fluidic element may be connected to commercially available syringes and/or needles), ease of use (e.g., conventional methods and equipment may be used for filling the conduits, in some cases), reduced or eliminated eccentricity between the fluid from the first conduit (e.g., a viscous fluid) and the fluid from the second conduit (e.g., a lubricating fluid) in the needle, coaxial lubrication, reduced splashback during injection, cost effectiveness, and/or increased feasibility of subcutaneous administration rather than intravenous administration. Examples of benefits that may arise from subcutaneous administration (which frequently require higher concentrations) rather than intravenous administration, in some embodiments, include increased feasibility of self-administration, reduced hospitalization, reduced treatment costs, and/or increased patient compliance.

In some embodiments, the articles and/or systems described herein can inject viscous fluids without the use of larger needle gauges or prolonged injection times, which can cause pain. Moreover, in certain embodiments, the articles and/or systems described herein can inject high concentration formulations without the use of syringe pumps, which can cause pain and can require a hospital setting. Additionally, in accordance with some embodiments, the articles and/or systems described herein can inject viscous fluids without the use of needle free jet injectors, which frequently result in contamination and high costs. Further, in accordance with certain embodiments, the articles and/or systems described herein can inject viscous fluids without particle encapsulation, which frequently results in protein inactivation, density based separation, needle clogging, and a higher degree of manufacturing complexity.

Various of the components described herein are “fluidically connected” to or “in fluidic communication” with other components. Generally, two components are fluidically connected and/or in fluidic communication when a connection exists between them such that fluid could flow and/or be transported from one to the other. In some cases, any two components that are described as “fluidically connected” or “in fluidic communication” may be directly fluidically connected or in direct fluidic communication, meaning that there are no components (e.g., a conduit or segment) between them. In certain instances, any two components that are described as “fluidically connected” or “in fluidic communication” may be indirectly fluidically connected or in indirect fluidic communication, meaning that there is one or more components (e.g., a conduit or segment) between them that does not prevent fluid from flowing and/or being transported from one to the other.

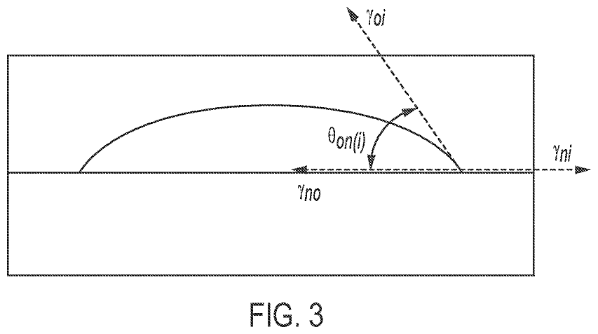

In some embodiments, the outer fluid (e.g., lubricating fluid and/or fluid from the second conduit) preferentially wets a surface (e.g., an interior surface of the needle and/or chamber) relative to the inner fluid (e.g., fluid of interest and/or fluid from the first conduit) when for the inner fluid, the outer fluid, and the surface, the spreading coefficient (Sₒₙ₍ᵢ₎) is greater than or equal to 0. FIG. 3 is a schematic illustration of a droplet of the outer fluid on the interior surface of the needle, where the outer droplet is surrounded by the inner fluid. The spreading coefficient can be determined according to the following equations:

$${S}_{on ( i )} = {γ}_{ni} - ( {γ}_{no} + {γ}_{oi} ) \qquad ( Equation 1 )$$

$$cos ( {θ}_{on ( i )} ) = \frac{{γ}_{ni} - {γ}_{no}}{{γ}_{oi}} \qquad ( Equation 2 )$$

$${S}_{on ( i )} = {γ}_{oi} ( cos ( {θ}_{on ( i )} ) - 1 ) \qquad ( Equation 3 )$$

In the equations above, gamma (γ) is the surface tensions of the various interfaces involved, where n is the subscript for a surface (e.g., an interior surface of the needle and/or chamber), o is the subscript for the outer fluid, and i is the subscript for the inner fluid. For example, γni denotes the surface tension between the surface and the inner fluid, γno denotes the surface tension between the surface and the outer fluid, and γoi denotes the surface tension between the outer fluid and the inner fluid. For example, in some embodiments, cos(θon(i)) and γoi are measured, and the spreading coefficient is determined by Equation 3. The spreading coefficient is specific to the three components (e.g., the interior surface of the needle, the inner fluid, and the outer fluid).

For the systems and methods described herein, the timescale of convection (T_{c}) is how long the inner fluid and outer fluid take to travel through the system (e.g., the needle and/or the chamber) while they are in direct contact with each other.

In some embodiments, the timescale of convection may be approximated by estimating the average volumetric flow rate of the multi-fluid system. In certain embodiments, the average volumetric flowrate and timescale of convection may be approximated using the following equations:

$${Q}_{avg} = \frac{{Q}_{i} + {Q}_{o}}{2} \qquad ( Equation 4 )$$

$${T}_{c} = \frac{L}{\overline{V}} = \frac{{LA}_{c}}{{Q}_{avg}} \qquad ( Equation 5$$

Where Q_{avg} is the average flowrate of the inner and outer fluids, Qi is the volumetric flowrate of the inner fluid, Qo is the volumetric flowrate of the outer fluid, L is the length of the system, A_{c} is the cross-sectional area of the system, and V̅ is the average linear velocity.

According to certain embodiments, LA_{c}/Q_{avg} is less than the timescale of eccentricity in the article. For example, in some embodiments, LA_{c}/Q_{avg} is less than or equal to 90%, less than or equal to 80%, less than or equal to 70%, less than or equal to 60%, or less than or equal to 50% of the timescale of eccentricity in the article. In certain cases, LA_{c}/Q_{avg} is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when LA_{c}/Q_{avg} is less than the timescale of eccentricity (tₑ), the fluids do not substantially exhibit eccentricity while in the system (e.g., the needle and/or article).

In certain embodiments, LA_{c}/Q_{avg} is less than the timescale of mixing (tₘ) in one or more portions of the system (e.g., in the needle and/or in the article) or in the entire system. For example, in some embodiments, LA_{c}/Q_{avg} is less than the timescale of mixing in the needle and/or LA_{c}/Q_{avg} is less than the timescale of mixing in the article. For example, in some embodiments, the ratio of LA_{c}/Q_{avg} for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when LA_{c}/Q_{avg} is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the LA_{c}/Q_{avg} and/or the ratio of LA_{c}/Q_{avg} to the timescale of eccentricity. For example, in accordance with certain embodiments, a ratio of LA_{c}/Q_{avg} to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of LA_{c}/Q_{avg} to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LA_{c}/Q_{avg} is less than

$$\sqrt{\frac{2 s}{\vert g cos ( θ ) ( 1 - \frac{{ρ}_{i}}{{ρ}_{o}} ) \vert}} .$$

For example, in some embodiments LA_{c}/Q_{avg} is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases, LA_{c}/Q_{avg} is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when LA_{c}/Q_{avg} is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In certain embodiments, LA_{c}/Q_{avg} is less than

$$\frac{{l}_{d}^{2}}{D i}$$

in one or more portions of the system (e.g., in the needle and/or in the article) or in the entire system. For example, in some embodiments, LA_{c}/Q_{avg} is less than

$$\frac{{l}_{d}^{2}}{D i}$$

in the needle and/or LA_{c}/Q_{avg} is less than

$$\frac{{l}_{d}^{2}}{D i}$$

in the article. For example, in some embodiments, the ratio of LA_{c}/Q_{avg} for the inner fluid and outer fluid to

$$\frac{{ι}_{d}^{2}}{D i}$$

for the inner fluid and outer fluid is less than or equal to 1, less than or equal to 0.75, less than or equal to 0.5, less than or equal to 0.1 or less than or equal to 0.01. In some embodiments, when LA_{c}/Q_{avg} is less than

$$\frac{{l}_{d}^{2}}{D i} ,$$

the fluids do not substantially mix while in the system or a portion thereof (e.g., the needle and/or article).

In some embodiments, the densities of the inner and outer fluids and/or the volumetric flow rate (Q) affects LA_{c}/Q_{avg} and/or the ratio of

$$\frac{{LA}_{c}}{{Q}_{avg}} to \sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{avg}} to \sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{avg}} to \sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LA_{c}/Q_{avg} is less than

$$\sqrt{\frac{2 s}{\vert g cos ( θ ) ( 1 - \frac{{ρ}_{o}}{{ρ}_{i}} ) \vert}} .$$

For example, in some embodiments LA_{c}/Q_{avg} is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases, LA_{c}/Q_{avg} is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when LA_{c}/Q_{avg} is less than

$$\sqrt{\frac{2 s}{\vert g cos ( θ ) ( 1 - \frac{{ρ}_{o}}{{ρ}_{i}} ) \vert}} ,$$

the fluids do not substantially exhibit eccentricity while in the system (e.g., the needle and/or article).

In some embodiments, the densities of the inner and outer fluids and/or the volumetric flow rate (Q) affects LA_{c}/Q_{avg} and/or the ratio of

$$\frac{{LA}_{c}}{{Q}_{a ν g}} to \sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{{LA}_{c}}{{Q}_{a ν g}} to \sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{{LA}_{c}}{{Q}_{a ν g}} to \sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In some embodiments, the timescale of convection may be approximated by estimating the total volumetric flow rate of the multi-fluid system. In certain embodiments, the total volumetric flowrate and timescale of convection may be approximated using the following equations:

$${Q}_{t o t o l} = {Q}_{i} + {Q}_{o} \qquad ( Equation 6 )$$

$${T}_{c} = \frac{L}{\overline{V}} = \frac{{LA}_{c}}{{Q}_{t o t a l}} \qquad ( Equation 7 )$$

Where Qₜₒₜₐₗ is the total volumetric flowrate of the inner and outer fluids, Qi is the volumetric flowrate of the inner fluid, Qo is the volumetric flowrate of the outer fluid, L is the length of the system, A_{c} is the cross-sectional area of the system, and V̅ is the average linear velocity.

According to certain embodiments, LA_{c}/Qₜₒₜₐₗ is less than the timescale of eccentricity in the article. For example, in some embodiments, LA_{c}/Qₜₒₜₐₗ is less than or equal to 90%, less than or equal to 80%, less than or equal to 70%, less than or equal to 60%, or less than or equal to 50% of the timescale of eccentricity in the article. In certain cases, LA_{c}/Qₜₒₜₐₗ is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when LA_{c}/Qₜₒₜₐₗ is less than the timescale of eccentricity (tₑ), the fluids do not substantially exhibit eccentricity while in the system (e.g., the needle and/or article).

In certain embodiments, LA_{c}/Qₜₒₜₐₗ is less than the timescale of mixing (tₘ) in one or more portions of the system (e.g., in the needle and/or in the article) or in the entire system. For example, in some embodiments, LA_{c}/Qₜₒₜₐₗ is less than the timescale of mixing in the needle and/or LA_{c}/Qₜₒₜₐₗ is less than the timescale of mixing in the article. For example, in some embodiments, the ratio of LA_{c}/Qₜₒₜₐₗ for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when LA_{c}/Qₜₒₜₐₗ is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the LA_{c}/Qₜₒₜₐₗ and/or the ratio of LA_{c}/Qₜₒₜₐₗ to the timescale of eccentricity. For example, in accordance with certain embodiments, a ratio of LA_{c}/Qₜₒₜₐₗ to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of LA_{c}/Qₜₒₜₐₗ to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LA_{c}/Qₜₒₜₐₗ is less than

$$\sqrt{\frac{2 s}{\vert g cos ( θ ) ( 1 - \frac{{ρ}_{i}}{{ρ}_{o}} ) \vert}} .$$

For example, in some embodiments LA_{c}/Qₜₒₜₐₗ is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases, LA_{c}/Qₜₒₜₐₗ is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when LA_{c}/Qₜₒₜₐₗ is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In certain embodiments, LA_{c}/Qₜₒₜₐₗ is less than

$$\frac{{l}_{d}^{2}}{D i}$$

in one or more portions of the system (e.g., in the needle and/or in the article) or in the entire system. For example, in some embodiments, LA_{c}/Qₜₒₜₐₗ is less than

$$\frac{{l}_{d}^{2}}{Di}$$

in the needle and/or LA_{c}/Qₜₒₜₐₗ is less than

$$\frac{{l}_{d}^{2}}{D i}$$

in the article. For example, in some embodiments, the ratio of LA_{c}/Qₜₒₜₐₗ for the inner fluid and outer fluid to

$$\frac{{l}_{d}^{2}}{Di}$$

for the inner fluid and outer fluid is less than or equal to 1, less than or equal to 0.75, less than or equal to 0.5, less than or equal to 0.1 or less than or equal to 0.01. In some embodiments, when LA_{c}/Qₜₒₜₐₗ is less than

$$\frac{{l}_{d}^{2}}{Di} ,$$

the fluids do not substantially mix while in the system or a portion thereof (e.g., the needle and/or article).

In some embodiments, the densities of the inner and outer fluids and/or the volumetric flow rate (Q) affects and/or the ratio of

$$\frac{{LA}_{c}}{{Q}_{total}} to \sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{total}} to \sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{total}} to \sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LA_{c}/Qₜₒₜₐₗ is less than

$$\sqrt{\frac{2 s}{\vert g cos ( θ ) ( 1 - \frac{{ρ}_{o}}{{ρ}_{i}} ) \vert}} .$$

For example, in some embodiments LA_{c}/Qₜₒₜₐₗ is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases, LA_{c}/Qₜₒₜₐₗ is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when LA_{c}/Qₜₒₜₐₗ is less than

$$\sqrt{\frac{2 s}{\vert g cos ( θ ) ( 1 - \frac{{ρ}_{o}}{{ρ}_{i}} ) \vert}} ,$$

the fluids do not substantially exhibit eccentricity while in the system (e.g., the needle and/or article).

In some embodiments, the densities of the inner and outer fluids and/or the volumetric flow rate (Q) affects LA_{c}/Qₜₒₜₐₗ and/or the ratio of

$$\frac{{LA}_{c}}{{Q}_{total}} to \sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{{LA}_{c}}{{Q}_{total}} to \sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{{LA}_{c}}{{Q}_{total}} to \sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In some embodiments, the timescale of convection may be approximated using the following equations:

$${T}_{c} = \frac{L}{\overline{V}} = \frac{LAi}{{Q}_{i}} \qquad ( Equation 8 )$$

where Ai may be estimated as shown below:

$${r}_{i}^{*} = \frac{{r}_{o}}{\sqrt{2 - {μ}_{o} / {μ}_{i}}} \qquad ( Equation 9 )$$

$${A}_{i} = {π ( {r}_{i}^{*} )}^{2} \qquad ( Equation 10 )$$

Where rᵢ* is the optimal radius of the inner fluid, μₒ is the dynamic viscosity of the outer fluid, μᵢ is the dynamic viscosity of the inner fluid, rₒ is the radius of the outer fluid, L is the length of the system, and Aᵢ is the cross-sectional area of the inner fluid as it flows through a region of interest of the system, and V̅ is the average linear velocity.

According to certain embodiments, LAᵢ/Qᵢ is less than the timescale of eccentricity in the article. For example, in some embodiments, LAᵢ/Qᵢ is less than or equal to 90%, less than or equal to 80%, less than or equal to 70%, less than or equal to 60%, or less than or equal to 50% of the timescale of eccentricity in the article. In certain cases, LAᵢ/Qᵢ is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when LAᵢ/Qᵢ is less than the timescale of eccentricity (tₑ), the fluids do not substantially exhibit eccentricity while in the system (e.g., the needle and/or article).

In certain embodiments, LAᵢ/Qᵢ is less than the timescale of mixing (tₘ) in one or more portions of the system (e.g., in the needle and/or in the article) or in the entire system. For example, in some embodiments, LAᵢ/Qᵢ is less than the timescale of mixing in the needle and/or A is less than the timescale of mixing in the article. For example, in some embodiments, the ratio of LAᵢ/Qᵢ for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when LAᵢ/Qᵢ is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the LAᵢ/Qᵢ and/or the ratio of LAᵢ/Qᵢ to the timescale of eccentricity. For example, in accordance with certain embodiments, a ratio of LAᵢ/Qᵢ to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of LAᵢ/Qᵢ to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LAᵢ/Qᵢ is less than

$$\sqrt{\frac{2 s}{\vert g cos ( θ ) ( 1 - \frac{{ρ}_{i}}{{ρ}_{o}} ) \vert}} .$$

For example, in some embodiments LAᵢ/Qᵢ is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases, LAᵢ/Qᵢ is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when LAᵢ/Qᵢ is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In certain embodiments, LAᵢ/Qᵢ is less than

$$\frac{{l}_{d}^{2}}{Di}$$

in one or more portions of the system (e.g., in the needle and/or in the article) or in the entire system. For example, in some embodiments, LAᵢ/Qᵢ is less than

$$\frac{{l}_{d}^{2}}{Di}$$

in the needle and/or LAᵢ/Qᵢ is less than

$$\frac{{l}_{d}^{2}}{Di}$$

in the article. For example, in some embodiments, the ratio of LAᵢ/Qᵢ for the inner fluid and outer fluid to

$$\frac{{l}_{d}^{2}}{Di}$$

for the inner fluid and outer fluid is less than or equal to 1, less than or equal to 0.75, less than or equal to 0.5, less than or equal to 0.1 or less than or equal to 0.01. In some embodiments, when LAᵢ/Qᵢ is less than

$$\frac{{l}_{d}^{2}}{D i} ,$$

the fluids do not substantially mix while in the system or a portion thereof (e.g., the needle and/or article).

In some embodiments, the densities of the inner and outer fluids and/or the volumetric flow rate (Q) affects LAᵢ/Qᵢ and/or the ratio of

$$\frac{{LA}_{i}}{{Q}_{i}} to \sqrt{\frac{2 s}{\vert g cos ( θ ) ( 1 - \frac{{ρ}_{i}}{{ρ}_{o}} ) \vert}} .$$

For example, in accordance with certain embodiments, a ratio

$$\frac{{LA}_{i}}{{Q}_{i}} to \sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{i}}{{Q}_{i}} to \sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, LAᵢ/Qᵢ is less than

$$\sqrt{\frac{2 s}{\vert g cos ( θ ) ( 1 - \frac{{ρ}_{o}}{{ρ}_{i}} ) \vert}} .$$

For example, in some embodiments LAᵢ/Qᵢ is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases, LAᵢ/Qᵢ is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when LAᵢ/Qᵢ is less than

$$\sqrt{\frac{2 s}{\vert g cos ( θ ) ( 1 - \frac{{ρ}_{o}}{{ρ}_{i}} ) \vert}}$$

the fluids do not substantially exhibit eccentricity while in the system (e.g., the needle and/or article).

In some embodiments, the densities of the inner and outer fluids and/or the volumetric flow rate (Q) affects LAᵢ/Qᵢ and/or the ratio of

$$\frac{L {A}_{i}}{{Q}_{i}} to \sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{L {A}_{i}}{{Q}_{i}} to \sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of to

$$\frac{L {A}_{i}}{{Q}_{i}} to \sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

For the systems and methods described herein, the timescale of eccentricity (tₑ) is the time for spatially stable eccentricity to arise in any part of the system (e.g., the needle, the chamber, and/or the article) comprising the inner fluid (e.g., first fluid and/or fluid from the first conduit) and outer fluid (e.g., second fluid and/or fluid from the second conduit).

In some embodiments, timescale of eccentricity may be approximated according to the following equation:

$${t}_{e} = \sqrt{\frac{2 s}{\vert g cos ( θ ) ( 1 - \frac{{ρ}_{i}}{{ρ}_{o}} ) \vert}} \qquad ( Equation 11 )$$

Where θ is the angle between the length of the needle and the horizontal plane, ρᵢ is density of the inner fluid, g is the gravitational constant and s is the displacement parameter (radial displacement of the centerline of the inner fluid from the axial centerline of the device), and ρₒ is density of the outer fluid.

According to certain embodiments, the timescale of convection is less than

$$\sqrt{\frac{2 s}{\vert g cos ( θ ) ( 1 - \frac{{ρ}_{i}}{{ρ}_{o}} ) \vert}}$$

in the article. For example, in some embodiments, the timescale of convection is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases, the timescale of convection is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when the timescale of convection (T_{c}) is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In some embodiments, the densities of the inner and outer fluids and/or the volumetric flow rate (Q) affects the timescale of convection and/or the ratio of the timescale of convection to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of timescale of convection to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of timescale of convection to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In certain embodiments, timescale of eccentricity may be approximated according to the following equation:

$${t}_{e} = \sqrt{\frac{2 s}{\vert g cos ( θ ) ( 1 - \frac{{ρ}_{o}}{{ρ}_{i}} ) \vert}} \qquad ( Equation 12 )$$

Where θ is the angle between the length of the needle and the horizontal plane, ρᵢ is density of the inner fluid, g is the gravitational constant and s is the displacement parameter (radial displacement of the centerline of the inner fluid from the axial centerline of the device), and ρₒ is density of the outer fluid.

According to certain embodiments, the timescale of convection is less than

$$\sqrt{\frac{2 s}{\vert g cos ( θ ) ( 1 - \frac{{ρ}_{o}}{{ρ}_{i}} ) \vert}}$$

in the article. For example, in some embodiments, the timescale of convection is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases, the timescale of convection is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when the timescale of convection (T_{c}) is less than

$$\sqrt{\frac{2 s}{\vert g cos ( θ ) ( 1 - \frac{{ρ}_{o}}{{ρ}_{i}} ) \vert}} ,$$

the fluids do not substantially exhibit eccentricity while in the system (e.g., the needle and/or article).

In some embodiments, the densities of the inner and outer fluids and/or the volumetric flow rate (Q) affects the timescale of convection and/or the ratio of the timescale of convection to

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of timescale of convection to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of timescale of convection to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In some embodiments, the orientation of the system (e.g., the needle and/or article) affects the timescale of eccentricity. For example, in accordance with certain embodiments, a T_{c}/tₑ of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments, the timescale of convection is less than the timescale of eccentricity in the article. For example, in some embodiments, the timescale of convection is less than or equal to 90%, less than or equal to 80%, less than or equal to 70%, less than or equal to 60%, or less than or equal to 50% of the timescale of eccentricity in the article. In certain cases, the timescale of convection is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when the timescale of convection (T_{c}) is less than the timescale of eccentricity (tₑ), the fluids do not substantially exhibit eccentricity while in the system (e.g., the needle and/or article).

In certain embodiments, the timescale of convection (T_{c}) is less than the timescale of eccentricity (tₑ). For example, in some embodiments, the ratio of the timescale of convection (T_{c}) for the inner fluid and outer fluid to the timescale of eccentricity (tₑ) for the inner fluid and outer fluid is less than or equal to 1, less than or equal to 0.75, less than or equal to 0.5, or less than or equal to 0.1. In some embodiments, when the timescale of convection (T_{c}) is less than the timescale of eccentricity (tₑ), the fluids do not substantially exhibit eccentricity while in the system (e.g., the needle and/or chamber).

For the systems and methods described herein, the timescale of mixing (tₘ) is the time needed for 50% of the outer fluid to mix with the inner fluid as they travel through the system or a portion thereof (e.g., the needle and/or the chamber) while they are in direct contact with each other. The timescale of mixing may be calculated using the following equation:

$${t}_{m} = \frac{{l}_{d}^{2}}{D i} \qquad ( Equation 13 )$$

Where Dᵢ is the diffusion coefficient of one or more components of the inner fluid (e.g., a drug (e.g., a biologic) in the inner fluid) in the outer fluid and I_{d} is the diameter of the part of the system (e.g., the needle and/or the chamber) where the fluids are in direct contact with each other. In embodiments where the system has portions with different diameters (e.g., a system comprising a chamber and a needle where the chamber has a larger diameter than the needle), the timescale of mixing may be determined using Equation 13 for each portion individually. In embodiments where the system has a varying geometry (e.g., if the chamber had an oval shape), the timescale of mixing may be determined using Equation 13 in conjunction with an integral approach.

In certain embodiments, the timescale of convection (T_{c}) is less than the timescale of mixing (tₘ) in one or more portions of the system (e.g., in the needle and/or in the chamber) or in the entire system. For example, in some embodiments, the timescale of convection is less than the timescale of mixing in the needle and/or the timescale of convection is less than the timescale of mixing in the chamber. For example, in some embodiments, the ratio of the timescale of convection (T_{c}) for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when the timescale of convection (T_{c}) is less than the timescale of mixing (tₘ), the fluids do not substantially mix while in the system or a portion thereof (e.g., the needle and/or chamber).

In some embodiments, the densities of the inner and outer fluids and/or the volumetric flow rate (Q) affects the timescale of convection and/or the ratio of the timescale of convection to the timescale of eccentricity. In accordance with certain embodiments, a T_{c}/tₑ of less than or equal to 1 is easier to achieve with smaller differences in density between the inner fluid and outer fluid and/or with a higher average volumetric flow rate of the inner fluid (Qᵢ).

In certain embodiments, when the outer fluid flow rate is too low compared to the inner fluid flow rate, a viscous displacement regime is observed rather than an axially lubricated flow regime. In a viscous displacement regime, the outer fluid fills the entire cross-section of the needle and forces both the inner fluid and the outer fluid to backflow into the outer fluid inlet. However, in certain cases, the backflow cannot be sustained due to the constant mass flux that is imposed on the outer fluid, resulting in a sudden overflow of the outer fluid into the needle. In some instances, this flow decreases until it is completely hindered once again, and the process repeats. In certain embodiments, this cyclic behavior results in unsteady and significantly worse lubrication compared to an axially lubricated flow regime.

In accordance with some embodiments, the ratio of the volumetric flow rate of the outer fluid (Qₒ) to the volumetric flow rate of the inner fluid (Qᵢ) is greater than 0.1. In some embodiments, the ratio of the volumetric flow rate of the outer fluid (Qₒ) to the volumetric flow rate of the inner fluid (Qᵢ) is greater than or equal to 0.2, greater than or equal to 0.4, or greater than or equal to 0.6. In certain embodiments, the ratio of the volumetric flow rate of the outer fluid (Qₒ) to the volumetric flow rate of the inner fluid (Qᵢ) is less than or equal to 1.

In some embodiments, the outer fluid and inner fluid do not mix substantially in the needle and/or chamber, because mixing dilutes the inner fluid, reducing the benefits of axially lubricated flow. In certain embodiments, the timescale of convection is shorter than the time it takes for the inner fluid and outer fluid to mix substantially in the needle and/or chamber. In accordance with some embodiments, the outer fluid mixes with the inner fluid at most 50% while in the needle and/or chamber. That is, at most 50% of the outer fluid is mixed with the inner fluid while in the needle and/or chamber while the remainder of the outer fluid remains unmixed with the inner fluid. For example, in certain embodiments, the outer fluid mixes with the inner fluid at most 40%, at most 30%, at most 20%, or at most 10% while in the needle and/or chamber. According to certain embodiments, the percentage of mixing can be determined by visual inspection. In some embodiments, this could be accomplished by dyeing the inner fluid and/or outer fluid, taking photographs at the outlet of the needle, and measuring the extent of mixing of the two fluids from the diffusion and/or spreading of the dye(s). In certain embodiments, the extent of mixing could be measured at different lengths by cutting the needle to the length of interest, and photographing the fluids at the outlet.

When the inner fluid and the outer fluid are in concentric contact and are moving, one or more components of the inner fluid (e.g., a drug, such as a biologic) may begin to diffuse into the outer fluid. The radial position of the distinction between the inner and outer fluids (R(x)) is given by the following equation:

$$R ( x ) = {R}_{0} + \sqrt{\frac{D x}{\overline{V}}} \qquad ( Equation 14 )$$

Where R₀ is the radius of the inner fluid at the beginning of any section of interest where the fluids are in contact, x is the axial position along the section, D is the diffusion coefficient of the component (e.g., a drug) in the outer fluid, and V̅ is the average velocity of the inner fluid. The extent of this diffusion can be validated by visualization as described elsewhere herein (e.g., by using dye molecules with the same diffusion coefficient as the component (e.g., drug)). As used herein, the radial position of the distinction between the inner and outer fluid (R(x)) means the distance between the center of the inner fluid and the distinction (e.g., boundary) between the inner and outer fluids. For example, when the inner fluid and the outer fluid first make contact and no diffusion has taken place, R(x) will be the same as R₀. However, as the fluids move through the system and the axial position (x) increases, R(x) will become larger than R₀, in some embodiments.

In some embodiments, it is beneficial for lower amounts of the outer fluid to be ejected compared to the amount of inner fluid ejected (e.g., such that a patient is not exposed to large amounts of a lubricating fluid). According to certain embodiments, the ratio of a volume of the inner fluid ejected from the needle to the total volume (e.g., inner fluid and outer fluid) ejected from the needle (Φ) (the volume fraction) is greater than or equal to 0.5, greater than or equal to 0.6, greater than or equal to 0.7, greater than or equal to 0.8, or greater than or equal to 0.9. The volume fraction (Φ) can also be expressed as:

$$Φ = {Q}_{i} / ( {Q}_{i} + {Q}_{o} ) \qquad ( Equation 15 )$$

In accordance with some embodiments, when the inner fluid has a certain capillary number and the outer fluid has a certain capillary number, axially lubricated flow may be observed, whereas viscous displacement may otherwise be observed. According to certain embodiments, the capillary number of the inner fluid is greater than or equal to 0.01, greater than or equal to 0.1, greater than or equal to 1, greater than or equal to 10, greater than or equal to 20, or greater than or equal to 25. In some embodiments, the capillary number of the inner fluid is less than or equal to 30, less than or equal to 25, less than or equal to 10, less than or equal to 1, or less than or equal to 0.1. Combinations of these ranges are also possible (e.g., greater than or equal to 0.01 and less than or equal to 30).

In some embodiments, the capillary number of the outer fluid is greater than or equal to 0.001, greater than or equal to 0.01, greater than or equal to 0.1, greater than or equal to 1, greater than or equal to 10, or greater than or equal to 20. In certain embodiments, the capillary number of the outer fluid is less than or equal to 25, less than or equal to 10, less than or equal to 1, less than or equal to 0.1, or less than or equal to 0.01. Combinations of these ranges are also possible (e.g., 0.001-25).

In certain embodiments, the capillary number of the inner fluid is larger than the capillary number of the outer fluid. The capillary number of a fluid is expressed as:

$${C}_{a} = \frac{( μ * V )}{σ} \qquad ( Equation 16 )$$

where μ (mu) is the dynamic viscosity of the fluid, V is the average linear velocity of the fluid, and σ (sigma) is the interfacial tension between the inner and outer fluids.

As discussed above, in some embodiments, it is beneficial for lower amounts of the outer fluid to be ejected compared to the amount of inner fluid ejected (e.g., such that a patient is not exposed to large amounts of a lubricating fluid). In certain embodiments, the volumetric flow rate of the inner fluid is greater than the volumetric flow rate of the outer fluid. According to some embodiments, the volumetric flow rate of the inner fluid is ≥10⁻²×γπdₙ²/μᵢ. For example, in certain cases, the volumetric flow rate of the inner fluid is ≥5×10⁻²×γπdₙ²/μᵢ or ≥10⁻¹×γπdₙ²/μᵢ. In accordance with certain embodiments, the volumetric flow rate of the outer fluid is ≥10⁻³×γπdₙ²/μ_{O}. For example, in some instances, the volumetric flow rate of the outer fluid is ≥10^{-3×}γπdₙ²/μ_{O}. For the volumetric flow rate, dₙ is the diameter of the needle, γ (gamma) is the surface tension of the two fluids, and μ is the dynamic viscosity of the fluid (where the i denotes the inner fluid and the o denotes the outer fluid).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

Unless clearly indicated to the contrary, the flowrates (Q) described herein are volumetric flow rates.

Unless clearly indicated to the contrary, the viscosities (μ) described herein are dynamic viscosities. The dynamic viscosity of a fluid can be determined using a TI ARG-2 rheometer, varying the shear rate from 10 s⁻¹ to 500 s⁻¹.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article for delivery of a fluid, comprising:
a first conduit comprising an inlet and an outlet;
a second conduit comprising an inlet and an outlet, the first conduit and the second conduit arranged in a side-by-side configuration; and
a chamber comprising:
a first internal volume;
a first inlet fluidically connected to the outlet of the first conduit and the first internal volume of the chamber;
a second internal volume that surrounds at least a portion of the first internal volume; and
a second inlet fluidically connected to the outlet of the second conduit and the second internal volume of the chamber.

2. The article of claim 1, further comprising:
a first plunger at least partially disposed in the first conduit; and
a second plunger at least partially disposed in the second conduit.

3. The article of claim 2, wherein the first plunger and the second plunger are connected by a solid body.

4. The article of claim 2, wherein the article is configured such that when the first plunger and the second plunger are compressed, fluid within the first conduit is transported to the chamber and fluid within the second conduit is transported to the chamber such that the fluid from the second conduit at least partially axially surrounds fluid from the first conduit.

5. The article of claim 2, wherein the article is configured such that when the first plunger and the second plunger are compressed, fluid within the first conduit is transported to the needle and fluid within the second conduit is transported to the needle such that the fluid from the second conduit at least partially axially surrounds fluid from the first conduit.

6. An article for delivery of a fluid, comprising:
a first conduit;
a first plunger associated with the first conduit;
a second conduit in a side-by-side configuration with the first conduit;
a second plunger associated with the second conduit;
a solid body connecting the first plunger and the second plunger; and
a chamber in fluidic communication with the first conduit and the second conduit;
wherein the article is configured such that when the first plunger and the second plunger are compressed, fluid within the first conduit is transported to the chamber and fluid within the second conduit is transported to the chamber such that the fluid from the second conduit at least partially axially surrounds fluid from the first conduit.

7. The article of claim 6, wherein the article is configured such that when the first plunger and the second plunger are compressed, fluid within the first conduit is transported to the needle and fluid within the second conduit is transported to the needle such that the fluid from the second conduit at least partially axially surrounds fluid from the first conduit.

8. The article of claim 6, wherein the chamber comprises a first internal volume and a second internal volume.

9. The article of claim 8, wherein:
the first conduit comprises an inlet and an outlet;
the second conduit comprises an inlet and an outlet;
the chamber comprises a first inlet fluidically connected to the outlet of the first conduit and the first internal volume of the chamber;
the chamber comprises a second inlet fluidically connected to the outlet of the second conduit and the second internal volume of the chamber; and
the second internal volume surrounds at least a portion of the first internal volume.

10. The article of claim 8, wherein the solid body is a cap connected to an end of the first plunger and an end of the second plunger.

11. The article of claim 1, wherein the chamber further comprises an outlet fluidically connected to the first internal volume and the second internal volume.

12. The article of claim 1, further comprising a needle in fluidic communication with the first internal volume and the second internal volume.

13. The article of claim 1, wherein:
the first conduit comprises a longitudinal axis;
the second conduit comprises a longitudinal axis; and
at least a portion of the longitudinal axis of the first conduit is within 10 degrees of parallel to at least a portion of the longitudinal axis of the second conduit.

14. The article of claim 1, wherein the first conduit and the second conduit are each at least partially contained within the same housing.

15. A fluidic element, comprising:
a first internal volume;
a second internal volume, wherein the second internal volume surrounds at least a portion of the first internal volume;
a first inlet within a surface of the fluidic element, the first inlet in fluidic communication with the first internal volume;
a second inlet within the surface of the fluidic element, the second inlet in fluidic communication with the second internal volume; and
an outlet in fluidic communication with the first internal volume and the second internal volume.

16. An article comprising the fluidic element of claim 15, and further comprising:
a first conduit;
a first plunger associated with the first conduit;
a second conduit in a side-by-side configuration with the first conduit;
a second plunger associated with the second conduit; and
a solid body connecting the first plunger and the second plunger;
wherein the fluidic element is in fluidic communication with the first conduit and the second conduit; and
wherein the article is configured such that when the first plunger and the second plunger are compressed, fluid within the first conduit is transported to the fluidic element and fluid within the second conduit is transported to the fluidic element such that the fluid from the second conduit at least partially axially surrounds fluid from the first conduit.

17. An article comprising the fluidic element of claim 15, and further comprising:
a first conduit comprising an inlet and an outlet; and
a second conduit comprising an inlet and an outlet, the first conduit and the second conduit arranged in a side-by-side configuration;
wherein the first inlet of the fluidic element is fluidically connected to the outlet of the first conduit and the first internal volume of the fluidic element;
wherein the second inlet of the fluidic element is fluidically connected to the outlet of the second conduit and the second internal volume of the fluidic element.
